(19) United States
(12) Patent Application Publication
(10) Pub. No.: US 2001/0016314 A1
(43) Pub. Date: Aug. 23, 2001

(54) LINKING GENE SEQUENCE TO GENE FUNCTION BY THREE DIMESIONAL (3D) PROTEIN STRUCTURE DETERMINATION

(76) Inventors: STEPHEN ANDERSON, PRINCETON, NJ (US); GAETANO MONTELIONE, HIGHLAND PARK, NJ (US)

Correspondence Address: JANE MASSEY LICATA, ESQ., LAW OFFICES OF JANE MASSEY LICATA, 66 E. MAIN STREET, MARLTON, NJ, 08053, US

(21) Appl. No.: 09/181,601

(22) Filed: Oct. 29, 1998

(51) Int. Cl. C12Q 1/68; C07H 21/02; C07H 21/04; C07K 14/00; C07K 1/00; G01N 33/50; G06F 19/00

(52) U.S. Cl. 435/6; 536/23.1; 530/350; 702/20

(57) ABSTRACT

The present invention provides a structure-functional analysis engine for the high-throughput determination of the biochemical function of proteins or protein domains of unknown function. The present invention uses bioinformatics, molecular biology and nuclear magnetic resonance tools for the rapid and automated determination of the three-dimensional structures of proteins and protein domains.

### CROSS-REFERENCE TO RELATED APPLICATIONS

This application claims priority under 119(e) to Provisional Patent Application No. 60/063,679, which was filed on Oct. 29, 1997.

### FIELD OF THE INVENTION

The present invention pertains to methods for elucidating the function of proteins and protein domains by examination of their three dimensional structure, and more specifically, to the use of bioinformatics, molecular biology, and nuclear magnetic resonance (NMR) tools to enable the rapid and automated determination of functions, as a means of genome analysis. The present invention further pertains to an integrated system for elucidating the function of proteins and protein domains by examining their three dimensional structure.

### BACKGROUND OF THE INVENTION

One of the most powerful ways of identifying the biochemical and medical function of a gene product is to determine its three-dimensional structure. Although there are numerous examples in which the primary (i.e., linear) structure of a protein has provided key clues to its biochemical function, three dimensional (3D) structure determination is considered to be more definitive at establishing biochemical function. The process of elucidating the 3D structure of large molecules, such as proteins is generally thought of as slow and expensive.

In the past, most drugs were discovered by screening proprietary chemicals with animal models or receptor libraries. Today, this approach is being replaced by “combinatorial chemistry” and “rational drug design”. These are the primary methods being used in the development of, for example, drugs targeted at the enzymes of the human AIDS virus.

What limits the drug discovery process today is not screening or medicinal chemistry but the rate that the approximately 100,000 proteins in the human body can be identified and prioritized as potential drug targets. Of particular significance for the pharmaceutical industry are the emerging disciplines of bioinformatics and functional genomics. Application of technologies developed in these areas will allow companies to identify, in the next decade, the bulk of the most significant new drug targets. It has been estimated that about 10,000 genes from the human genome are of potential value in human medicine, but only a few percent of these genes have been isolated so far. However, it is reported that by the year 2005 the raw sequence data for all of these genes will have been determined by the Human Genome Project (HGP).

### I. PROTEIN STRUCTURE

It is a generally accepted principle of biology that a protein's primary sequence is the main determinant of its tertiary structure. Anfinsen, Science 181:223-230 (1973); Anfinsen and Scheraga, Adv. Prot. Chem. 29:205-300 (1975); and Baldwin, Ann. Rev. Biochem. 44:453-475 (1975). For over a decade, researchers have been studying the theoretical and practical aspects of the folding of recombinant proteins.

For example, the “genetics” of protein folding using mutants of bovine pancreatic trypsin inhibitor (BPTI) has been studied. Mutants of BPTI were prepared in which several cysteine residues were replaced by alanine or threonine residues. These mutants were then expressed in a heterologous E. coli expression system. Although these mutants were found to fold into the proper conformation, the rate of the mutant folding was somewhat slower than that exhibited by wild-type BPTI. Marks et al., Science 325:1370-1373 (1987).

Ma et al have also studied the genetics of protein folding using mutants of BPTI. Ma et al., Biochemistry 36:3728-3736 (1997). The model system described by Ma et al. predicts that a “rearrangement” mechanism to form buried disulfides at a late stage in the folding reaction may be a common feature of redox folding pathways for surface disulfide-containing proteins of high stability.

Nilsson et al. have reported that factors, such as peptidyl prolyl isomerase, protein disulfide isomerase, thioredoxin, and Sec B, may interact with the unfolded forms of specific classes of proteins, while members of the hsp70/DnaK and hsp60/GroEL molecular chaperone families may play a more general role in protein folding. Nilsson et al., Ann. Rev. Microbiol. 45:607-635 (1991). Nilsson et al. further disclose that intrinsic folding rates, or even translation rates, of nascent proteins may be optimized by natural selection. Secretion, proteolysis and aggregation are other in vivo processes that depend greatly in the folding behavior of a given protein. Thus, protein folding involves an interplay between the intrinsic biophysical properties of a protein, in both its folded and unfolded states, and various accessory proteins that aid in the process.

Proteins are generally composed of one or more autonomously-folding units known as domains. Kim et al., Ann. Rev Biochem. 59:631-660 (1990); Nilsson et al., Ann. Rev. Microbiol. 45:607-635 (1991). Multidomain proteins in higher organisms are encoded by genes containing multiple exons. Combinatorial shuffling of exons during evolution has produced novel proteins with different domain arrangements having different associated functions. This is thought to have greatly increased the ability of higher organisms to respond to environmental challenges because, via recombinational events, it has enabled genomes to readily add, subtract, or rearrange discrete functionalities within a given protein. Patthy, Cell 41:657-663 (1985); Patthy, Curr. Opin. Struct. Bio. 4:383-392 (1994); and Long et al., Science 92:12495-12499 (1995).

### II. INTERPRETATION OF A PROTEIN STRUCTURE

Several methods have been used to elucidate the 3D structure of a given protein molecule. Chiefly, these methods are X-ray crystallography and Nuclear Magnetic Resonance (NMR).

A. X-ray crystallography

X-ray crystallography is a technique that directly images molecules. A crystal of the molecule to be visualized is exposed to a collimated beam of monochromatic X-rays and the consequent diffraction pattern is recorded on a photographic film or by a radiation counter. The intensities of the diffraction maxima are then used to construct mathematically the three-dimensional image of the crystal structure. X-rays interact almost exclusively with the electrons in the matter and not the nuclei.

The spacing of atoms in a crystal lattice can be determined by measuring the angle and intensities at which a beam of X-rays of a given wave length is diffracted by the electron shells surrounding the atoms. Operationally, there are several steps in X-ray structural analysis. The amount of information obtained depends on the degree of structural order in the sample. Blundell et al. provide an advanced treatment of the principles of protein X-ray crystallography. Blundell et al., Protein Crystallography, Academic Press (1976), herein incorporated by reference. Likewise, Wyckoff et al. provide a series of articles on the theory and practice of X-ray crystallography. Wyckoff et al. (Eds.), Methods Enzymol. 114: 330-386 (1985), herein incorporated by reference.

B. Nuclear Magnetic Resonance (NMR)

The classical approach for the analysis of NMR resonance assignments was first outlined by Wüthrich, Wagner and co-workers. Wüthrich, “NMR of proteins and nucleic acids” Wiley, New York, New York (1986); Wüthrich, Science 243:45-50 (1989); Billeter et al., J. Mol Biol. 155:321-346 (1982), all of which are herein incorporated by reference. For a general review of protein determination in solution by nuclear magnetic resonance spectroscopy, see Wüthrich, Science 243:45-50 (1989). See also, Billeter et al., J. Mol. Biol. 155:321-346 (1982).

Wüthrich's classical approach can be briefly summarized in the following seven steps:

Step 1: Identification of individual resonances associated with each spin system, and designation of key atom types (e.g., H^{N}, H^{α}, N, C^{α}, C^{β}, etc.).

Step 2: Classification of each identified spin system with respect to one or more possible amino acid residue type(s).

Step 3: Identification of possible sequential relations between spin systems using inter-residue NOESY or triple-resonance data.

Step 4: Unique mapping of strings of sequentially-connected spin systems to segments of the amino acid sequence, thus establishing “sequence specific assignments.”

Step 5: Extension of assignments to resonances of peripheral side-chain nuclei in each spin system, and determination of stereospecific assignments.

Step 6: Generation of distance constraints using assigned resonance frequencies to interpret NOESY, scalar-coupling, and hydrogen/deuterium-exchange data in terms of “sequence-specific distance constraints.”

Step 7: Structure generation using these constraints.

Automated implementation of these methods have made use of exhaustive search, constraint satisfaction, heuristic best-fit or branch-and-bound limited search, genetic, neural net, pseudoenergy minimization, and simulated annealing satisfaction. Billeter et al., J. Magn. Resonance 76:400-415 (1988); Zimmerman et al., In: Proceedings of the First International Conference of Intelligent Systems for Molecular Biology. Washington: AAAS Press (1994); Zimmerman et al., J. Biomol NMR 4:241-256 (1994); Zimmerman et al., Curr. Opin. Struct. Bio. 5:664-673 (1995); and Zimmerman et al, J. Mol. Bio. 269:592-610 (1997).

Under traditional methodology, before a given protein is studied at the 3D level, the researcher had already obtained detailed experimental information regarding the protein's function and characteristics. The 3D structure is typically the last of many experiments performed over many years of study. The 3D structure information is then used to refine the researcher's understanding of the given protein. Thus, under traditional methodology, it is very rare that the 3D structure of a given protein is determined before its biochemical function has been determined by other methods.

The present invention represents a paradigm shift in methodology because the researcher would first determine the 3D structure of a protein of unknown function and then use this structure to gain clues as to its function, which would be subsequently validated by appropriate biochemical assays.

### SUMMARY OF THE INVENTION

The present invention describes an integrated system for rapid determination of the three-dimensional structures of proteins and protein domains and application of this technology in a high-throughput analysis of human and other genomes for drug discovery purposes.

The “structure-function analysis engine” described herein has the potential to discover the functions of novel genes identified in the human and other genomes faster than existing genetic or purely computational bioinformatics methods.

The present invention employs:

1. Bioinformatics methods, including the analysis of exon-exon phases and other methods for segmenting or “parsing” DNA sequences of novel genes into domain-encoding regions;

2. Robust and general “domain trapping” methods for producing correctly-folded recombinant protein domains of novel biomedically-important human disease gene products;

3. Robust and general methods for high level expression and isotopic enrichment of these domains for NMR and X-ray crystallographic studies;

4. Screening methods to identify protein domain constructs that exhibit the properties required for structural analysis by NMR or X-ray crystallography;

5. Computer software, NMR pulse sequences, and related NMR technologies that provide fully automated analysis of protein structures from NMR data;

6. NMR spectroscopy methods for determining 3D structures of these domains;

7. Improved methods for mapping new domain structures to proteins in the Protein Data Bank that have similar structures and biochemical functions;

8. A relational data base of the empirical properties of expressed domains for organizing and integrating the biophysical and biological information derived from these studies, as well as methods for making such relational data bases; and

9. A method for integrating all of the above into a large-scale, high-throughput macromolecular “structure-function analysis engine,” and the application this “structure-function analysis engine” to the discovery of biochemical functions of hundreds of genes from humans and human pathogens.

The specific biomedical gene targets that this technology can be used to develop include:

1. Domains from the human Alzheimer's β peptide precursor protein (APP).

2. Domains from other proteins genetically implicated in neoplastic, metabolic, neurodegenerative, cardiovascular, psychiatric and inflammatory disorders.

3. Domains from proteins associated with infectious agents (e.g., bacteria, fungi and viruses).

The present invention provides a high-throughput method for determining a biochemical function of a protein or polypeptide domain of unknown function comprising: (A) identifying a putative polypeptide domain that properly folds into a stable polypeptide domain, the stable polypeptide having a defined three dimensional structure; (B) determining three dimensional structure of the stable polypeptide domain; (C) comparing the determined three dimensional structure of the stable polypeptide domain to known three-dimensional structures in a protein data bank, wherein the comparison identifies known structures within the protein data bank that are homologous to the determined three dimensional structure; and (D) correlating a biochemical function corresponding to the identified homologous structure to a biochemical function for the stable polypeptide domain.

The present invention further provides an integrated system for rapid determination of a biochemical function of a protein or protein domain of unknown function: (A) a first computer algorithm capable of parsing the target polynucleotide into at least one putative domain encoding region; (B) a designated lab for expressing the putative domain; (C) an NMR spectrometer for determining individual spin resonances of amino acids of the putative domain; (D) a data collection device capable of collecting NMR spectral date, wherein the data collection device is operatively coupled to the NMR spectrometer; (E) at least one computer; (F) a second computer algorithm capable of assigning individual spin resonances to individual amino acids of a polypeptide; (G) a third computer algorithm capable of determining tertiary structure of a polypeptide, wherein the polypeptide has had resonances assigned to individual amino acids of the polypeptide; (H) a database, wherein stored within the database is information about the structure and function of known proteins and determined proteins; and (I) a fourth computer algorithm capable of determining 3D structure homology between the determined three-dimensional structure of a polypeptide of unknown function to three-dimensional structure of a protein of known function, wherein the protein of known structure is stored within the protein database.

The present invention further provides a high-throughput method for determining a biochemical function of a polypeptide of unknown function encoded by a target polynucleotide comprising the steps: (A) identifying at least one putative polypeptide domain encoding region of the target polynucleotide (“parsing”); (B) expressing the putative polypeptide domain; (C) determining whether the expressed putative polypeptide domain forms a stable polypeptide domain having a defined three dimensional structure (“trapping”); (D) determining the three dimensional structure of the stable polypeptide domain; (E) comparing the determined three dimensional structure of the stable polypeptide domain to known three dimensional structures in a Protein Data Bank to determine whether any such known structures are homologous to the determined structure; and (F) correlating a biochemical function corresponding to the homologous structure to a biochemical function for the stable polypeptide domain.

### BRIEF DESCRIPTION OF THE FIGURES

FIG. 1 provides a flow chart of the high-throughput structure/function analysis system of the present invention.

FIG. 2A provides the far UV circular dichroism spectra of the purified recombinant APP NTD2-3 domain. FIG. 2B provides the near UV circular dichroism spectra of the purified recombinant APP NTD2-3 domain.

FIG. 3 provides a NMR spectra of the purified recombinant APP NTD2-3.

FIG. 4 provides a hydrogen-deuterium exchange time course for the purified recombinant APP NTD2-3.

FIG. 5 provides the results of a cooperative thermal unfolding experiment of the purified recombinant APP NTD2-3.

FIG. 6 provides the results of the NMR ¹⁵N-¹H heteronuclear single quantum coherence (HSQC) spectral analysis of the NTD2-3 domain collected on a Varian Unity 500 spectrometer.

FIG. 7 provides the 2D ¹⁵N-¹H^{N} HSQC spectrum of CspA at pH 6.0 and 30° C.

FIG. 8A provides an illustration of information derived from triple resonance data sets used for establishing intraresidue and sequential correlations of spin systems.

FIG. 8B provides an illustration of NMR data used to identify structural elements in CspA. Slowly exchanging backbone amides (t_{½}>3 min at pH 6.0 and 30° C.) are indicated by filled circles (t_{½}<30 min) or starts (t_{½}>30 min.). Values of ³J(H^{N}-H^{α}) coupling constants are indicated by vertical bars; filled bars indicate that the data provided a useful estimate (±0.5 Hz) of the corresponding coupling constant, while open bars indicate that the experimental data provide only an upper bound on its value. Values of conformation-dependent secondary shifts ΔδC^{α} and ΔδC^{β} are plotted with solid bars. The locations of the five β-strands are indicated with arrows.

### DESCRIPTION OF THE PREFERRED EMBODIMENTS

One of the best clues to a protein's function is its structure. The present invention describes a structure-based bioinformatics platform to be used in “functional genomics” analyses of the torrent of DNA sequence data emerging from the international HGP. This technology will allow for the isolation of novel biopharmaceuticals and/or drug targets from gene sequence information with an efficiency that is far beyond present day capabilities. By developing extremely fast yet rigorous technologies for macromolecular structure determination, it is possible to convert the stream of one-dimensional DNA sequence information emerging from human genome research efforts into 3D protein structures. This 3D structural information can then be used to map these human gene products to protein families with similar biochemical functions.

The present invention describes a “drug discovery search engine” that allows human genetic and genomic data to be smoothly interfaced with proven rational drug design and combinatorial chemistry approaches. The technology described herein enables determination of the structures for virtually the entire complement of human protein domains, encoded in the approximately 100,000 human genes.

### III. STRUCTURE SUGGESTS FUNCTION

It is a tenet of modem structural biology that structure suggests function: a given protein “fold” tends to be used over and over again in nature for a restricted set of biological functions. Knowledge of the structure of a new protein often reveals kinship to a family of other proteins with already known functions, and thus provides strong clues regarding the biochemical function of the protein at hand. Holm et al., Science 273:595-603 (1996); Bork et al., Curr. Opin. Struct. Bio. 4:393-403 (1994); Brenner et al., Proc. Natl. Acad. Sci. (U.S.A). 95:6073-6078 (1998), all of which are herein incorporated by reference. This kinship relationship is a natural manifestation of the fact that families of protein molecules have evolved from a common ancestral molecule, and that in the course of this evolution the 3D structure is largely preserved while new, though chemically related, biochemical functions are adopted. This is precisely the reasoning behind the assigning of “expressed sequence tag” (EST) sequences to known protein families using one-dimensional sequence comparisons.

Evolution generally acts to conserve 3D structures rather than the amino acid sequences of proteins. For this reason, proteins have often evolved over time so that their sequences exhibit no obvious similarity while their structures remain highly homologous. In practical terms, this means that simple sequence comparisons overlook many—and perhaps even most—instances of protein-protein relatedness. However, this relatedness, with all of its functional implications, can easily be identified by 3D structure comparisons.

The multidomain nature of many mammalian proteins makes them more difficult to express in recombinant form and also impedes their structure determination by X-ray crystallography or NMR. The expression and structure determination of an isolated domain is, in contrast, less problematical. Since an isolated domain comprises one or more discrete functional units in a protein, knowing structure-function information about a given individual domain in a multicomponent protein generally provides key information that can be used to proceed with drug development on the full-length protein. The “domain trapping” methods of the present invention generate many novel gene products suitable for structural analysis by NMR spectroscopy and X-ray crystallography.

Recent developments in the areas of high-level protein expression technology, X-ray crystalography, heteronuclear NMR spectroscopy, and artificial intelligence (Al)-based structural analysis software, have dramatically improved the speed and lowered the cost of protein structure determination. Estimates of the total number of human genes in the genome (approximately 10⁵) contrast dramatically with estimates of the total number of protein folds in nature (approximately 10³), and it has been estimated that one-third to one-half of these folds have already been described. Chothia et al., Nature 357:543-544 (1992). Simple statistics imply that many new gene products will exhibit structures that map to existing fold classes associated with proteins of known biochemical function. Thus, the harvest of functional information about new human genes from this approach will be immediate.

### IV. DESIGN OF A HIGH-THROUGHPUT SYSTEM FOR DETERMINING PROTEIN STRUCTURES AND FUNCTIONS

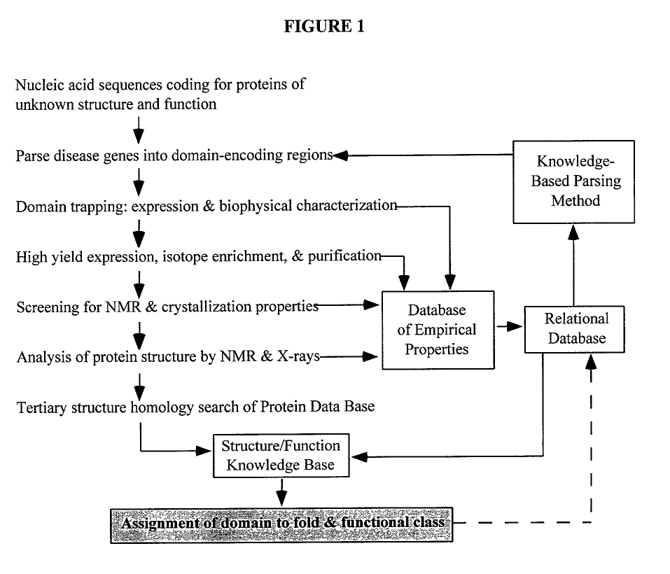

FIG. 1 provides a flow chart of the high-throughput structure/function analysis used in the present invention for analyzing human and pathogen gene products. This flow chart outlines the general methods of the present invention. Each sub-step of the present invention is outlined in detail below. It is to be understood that the hardware disclosed herein can be or is operatively linked to one or more computers.

A. Approaches for identifying novel protein domains

The present invention provides a method for predicting the location of domains and domain boundaries within a given DNA sequence. Under one embodiment, this is accomplished through a knowledge based application which segments or “parses” genomic or cDNA sequences of genes into domain encoding sequences. Under another embodiment, the knowledge based application of the present invention can also segment or “parse” MRNA sequences into domain encoding sequences. Preferably, the knowledge based application of the present invention is encoded within a computer algorithm software application. Preferably, this expert system applies rules developed on a set of experimentally-verified DNA sequence/protein domain comparisons that have been compiled from public sequence and protein structure databases. Thus, for a novel gene sequence, this expert system generates the predicted domains and/or domain boundaries which are then used to create domain-specific expression constructs.

Under one of the preferred embodiments, the gene sequence is parsed by the exon phase rule. Exon termini (5′- or 3′) that begin or end within protein coding regions can be classified according to their “phase”: an exon terminus that falls between two codons is called a “phase 0” terminus; an exon terminus that starts or stops after the first nucleotide in the codon is called a “phase 1” terminus; and an exon terminus that starts or stops after the second nucleotide in the codon is called a “phase 2” terminus. For example, where (“*”) marks the positions of an exon-exon junction--

```
Phase 0: *
5′ . . . -+E,uns A-T-G-+E,uns G-G-A-+E,uns C-T-C- . . . 3′
      . . . - Met - Gly - Leu - . . .
Phase 1: *
5′ . . . -+E,uns A-T-G-+E,uns G-G-A-+E,uns C-T-C- . . . 3′
      . . . - Met - Gly - Leu - . . .
Phase 2: *
5′ . . . -+E,uns A-T-G-+E,uns G-G-A-+E,uns C-T-C- . . . 3′
      . . . - Met - Gly - Leu - . . .
```

The genetic coding sequences for protein domains, which have been reported to have been “shuffled” between various genes during evolution, should be bounded by exon termini of the same phase (or by the N- or C-terminal ends of the holoprotein), otherwise insertion of these domains into a host gene would result in a frame-shift mutation in the downstream sequences upon splicing (Patchy, Cell 41:657-663 (1985); Patthy, FEBS Letters 214:1-7 (1987); Patthy, Cur. Opin. Struct. Bio. 4:383-392 (1994), all of which are herein incorporated by reference). Therefore, the domain encoding regions should be bounded on both sides by phase 0 exon termini, by phase 1 exon termini, or by phase 2 exon termini, but not by termini of different phases.

As part of the mechanism of molecular evolution, structural and functional domains are mixed and matched between protein sequences through the processes of gene duplication and crossover. Accordingly, under one preferred embodiment domains are identified by looking for segments of gene sequences that are conserved across many genes from different organisms. Known domain families generally involve 50 -300 amino-acid long segments that are observed as portions of many different proteins. Bioinformatics algorithms capable of identifying these conserved segments, or gene-fragment clusters, in the data base of gene sequences have been reported. These algorithms can be used to identify candidate domain-encoding regions in novel gene sequences. Gouzey et al., Trends Biochem. Sci. 21:493 (1994), herein incorporated by reference.

Under a second preferred embodiment, domains from gene sequence data are identified through predictions of their interdomain boundaries. There is ample evidence from molecular evolution and cell biology studies that information regarding domain boundaries is embedded in the sequences of protein coding genes. Some reports have claimed that rare codon clusters, which cause ribosomal pausing during translation, are correlated with domain boundaries. Purvis et al., J. Mol. Biol. 193:413-417 (1987); Nilsson et al., Ann. Rev. Microbiol. 45:607-635 (1991); Thanaraj et al., Protein Sci. 5:1973-1983 (1996); Thanaraj et al., Protein Sci. 5:1594-1612 (1996); and Guisez et al., J. Theor. Biol. 162:243-252 (1993), all of which are herein incorporated by reference. Messenger RNA secondary structure have also been reported to play such a “punctuation” role during translation.

One embodiment of the present invention employs an algorithm that identifies such sequence features and compares these data with the actual domain sequences in the relational database of the present invention. The relational database of the present invention contains domain sequence information of known and determined protein domains. It is understood that the relational database of the present invention will expand over time such that each polypeptide domain determined using the methods of the present invention will be added to the relational database. Under this embodiment, it is possible to rigorously assess the reliability of these bioinformatics methods of domain prediction and, iteratively, modify the software to improve its reliability. Neural nets and genetic algorithms both can be used for deriving rules for domain boundaries from this knowledge base. This invention markedly accelerates productivity by greatly reducing the number of expression constructs that would have to be tested in order to correctly parse a novel gene sequence into its component domain sequences.

Under another embodiment, the solution structure of a protein or protein domain can be analyzed by a method that combines enzymatic proteolysis and matrix assisted laser desorption ionization mass spectrometry (Cohen et al., Protein Sci. 4:1088-1099 (1995), Seielstad et al., Biochem. 34:12605-12615 (1995), both of which are incorporated by reference in their entirety). This method is capable of inferring structural information from determinations of protection against enzymatic proteolysis as governed by solvent accessibility and protein flexibility. Preferably, the proteolic enzymes employed by this method include trypsin, chymotripsin, thermolysin, and ASP-N endoprotease.

B. “Domain Trapping”: Expression and biophysical characterization of putative recombinant protein domains

With respect to genes of unknown function, the investigator, generally, does not have available an enzyme assay or other obvious activity-based means to assess the biochemical activity of a novel recombinant protein domain The present invention has addresses this difficulty in a three-pronged manner. First, the present invention uses a reliable and high yield expression system for protein expression. For example, a secretion-based protein A fusion system that is one of the most tested and reliable methods known for producing correctly-folded recombinant proteins in the E. coli periplasm. Nilsson et al., Methods Enzymol. 185:144-161 (1990), herein incorporated by reference. Alternatively, the pET plasmid expression system may be used. Studier et al., J. Mol. Bio. 189:113-130 (1986), herein incorporated by reference. Second, the present invention uses a set of activity-independent biophysical criteria to assess whether the protein domain has properly folded. This set of criteria has been developed through extensive study of recombinantly-expressed protein folding mutants. Finally, based on the supposition that autonomous folding of the protein domain can be prevented due to too much or too little polypeptide sequence information, respectively, (Kim et al., Ann. Rev. Biochem. 59:631-660 (1990); Nilsson et al., Ann. Rev. Microbiol. 45:607-635 (1991), both of which are herein incorporated by reference), the present invention uses systematic strategies for identifying and trapping domains that enables it to use a combination of molecular biological and biophysical methods to experimentally parse any gene into its component domains. In other words, a polypeptide domain has a “defined three dimensional structure” when that polypeptide domain exhibits the activity-independent biophysical criteria of a properly folded domain.

Under one preferred embodiment, an activity-independent biophysical criteria used to assess the correctness of folding of a protein includes circular dichroism measurements. More preferably, characterization of an isolated domain of a protein is analyzed by circular dichroism measurements in the far UV. An ellipticity minimum at 222 nm is indicative of α-helical secondary structure. Preferably, CD measurements at longer wavelengths are also determined (for a general review of CD and other methods, see Creighton, Proteins: Structure and molecular properties, 2nd Ed., W. H. Freeman & Co., New York, New York (1993, and related texts), herein incorporated by reference). A signal in the aromatic region around 280 nm is consistent with the presence of Trp, Tyr, and Phe chromophores in an ordered environment, such as would be expected in the hydrophobic core of a folded protein. In general, assays for the affinity-purified expressed proteins that employ solely biophysical criteria have been designed based upon experience with the behavior of misfolded recombinant proteins.

It is preferable to further characterize the isolated domain by ¹H-NMR spectroscopy. Preferably, the isolated domain is in a moderately concentrated solution (˜100 μM). A high dispersion pattern of the proton resonance spectrum is reported to be characteristic of a well-folded polypeptide.

A time-course of amide hydrogen-deuterium exchange measurements can also be performed on the isolated domain. From this, it is possible to observe whether backbone NH groups are significantly protected within the domain. Significant protection is an indication that the hydrogen-bonded secondary structure is stabilized by tertiary interactions, which is consistent with a well-folded domain structure.

Finally, thermal denaturation experiments, monitored by intrinsic tryptophan fluorescence, can also be performed. These experiments are also capable of determining whether the isolated domain is a compact domain structure.

In principle, this is a general strategy. Thus, it can be used to parse many genes in the human genome that encode proteins of unknown biochemical function into their component domains and express correctly-folded polypeptide for structure/function studies. This general strategy can be easily modified to provide a high-throughput method for validating candidate domains identified by the bioinformatics methods of the present invention. For a typical 10-30 kD protein domain, 500 or 600 MHz one-dimensional (ID) NMR spectra can be obtained in tens of minutes using only small quantities (˜200 μg) of protein. Using a continuous flow NMR probe with a microcomputer-controlled chromatography pump and simple sample changer, it is possible to automatically screen 50 - 100 candidate domains per day for folded structure. Those candidate domains which exhibit chemical shift dispersion indicative of ordered domain structure can then be further validated using the other biophysical techniques described above. An NMR spectrometer suitable for use in the present invention is a Varian Unity 500 spectrometer.

C. High level expression and isotopic enrichment

Uniform biosynthetic enrichment with ¹⁵N, ¹³C and ²H isotopes has been reported to be a prerequisite for the analysis of macromolecular structures by NMR spectroscopy. Some NMR strategies have also been reported to benefit from random enrichment with ²H isotopes. The principal obstacle for isotope-enriched protein production in most recombinant production systems is the high cost of the enriched media components (e.g. ¹³C-glucose @$330/g), and the limiting possibilities for scale-up to controlled multi-liter fermenters. The less well-controlled conditions of shaker flask cultivations often result in lower protein production levels. The production of ¹⁵N-,¹³C-, and/or ²H-enriched proteins thus requires an efficient system cable of providing high level production of the desired protein in small-scale bioreactors.

Under one preferred embodiment, the present invention employs a bacterial production system for ¹⁵N, ¹³C-enriched recombinant proteins. Preferably, the bacterial production system is based on intracellular production of recombinant proteins in E. coli as fusions to an IgG-binding domain analogue, Z, derived from staphylococcal Protein A (Nilsson et al., Protein Eng. 1:107-113 (1987); Altman et al., Protein Eng. 4:593-600 (1991), both of which are herein incorporated by reference). In this system, transcription is initiated from the efficient promoter of the E. coli trp operon. This allows for efficient intracellular production of fusion proteins. These fusion proteins can then be purified by IgG affinity chromatography. Using this approach it is possible to achieve high-level (40-200 mg/L) production in defined minimal media of a number of isotope-enriched proteins (see, for example, Jansson et al., J. Biomol. NMR 7:131-141 (1996)).

Under another preferred embodiment, the recombinant isotope-enriched domain protein may be produced using pET plasmid expression vectors (Studier et al., J. Mol. Biol. 189:113-130 (1986), herein incorporated by reference) under the control of the T7 RNA polymerase promoter (see, for example, Newkirk et al., Proc. Nat'l Acad. Sci. (U.S.A) 91:5114-5118 (1994); Chateijee et al., J. Biochem. 114:663-669 (1993); and Shimotakahara et al., Biochemistry 36:6915-6929 (1997), all of which are herein incorporated by reference).

Under another preferred embodiment, ¹⁵N, ¹³C, ²H-enriched recombinant proteins can be produced by acclimating a bacterial production system to grow in 95% ²H₂O. Recombinant bacterial production hosts [e.g., the BL21 (DE3) strain] can be acclimated to grow in 95% ²H₂O by successive passages in media containing increasing amounts of ²H₂O; protein production levels of acclimated bacteria grown in 95% ²H₂O are identical to those obtained in H₂O. Using protiated [uniformly ¹³C-enriched]-glucose as the carbon source, ²H-enrichment levels of 70 - 80% can be achieved; high incorporation of ²H from the ²H₂O solvent results from metabolic shuffling during amino acid biosynthesis. While the resulting proteins are not 100% perdeuterated, they are sufficiently enriched for the purpose of slowing ¹³C transverse relaxation rates and enhancing the sensitivity for certain types of triple-resonance NMR experiments. 100% perdeuterated samples can also be produced using ²H₂O solvent and [uniformly 21H, ¹³C-enriched]-glucose as the carbon source.

Under one preferred embodiment, such isotope enriched proteins can be renatured by the method of Kim et al. which employs in situ refolding of proteins immobilized on a solid support. Kim et al., Prot. Eng. 10:445-462 (1997), herein incorporated by reference. The isotope enriched proteins can also be renatured by the method of Maeda et al. which employs programmed reverse denaturant gradients. Maeda et al., Protein Eng. 9:95-100 (1996); Maeda et al., Protein Eng. 9:461-465 (1996), both of which are herein incorporated by reference. Under another preferred embodiment, the method of Kim et al. is coupled with the method of Maeda et al. Under yet another preferred embodiment, “active” folding agents, such as the molecular chaperones GroEL/ES, dnaK, dnaJ, etc., may be used to assist in protein folding. Nilsson et al., Ann. Rev. Microbiol. 45:607-635 (1991), herein incorporated by reference.

Preferably, the fusion vectors are constructed to interface with downstream refolding operations. Such vectors permit, for example, the binding of fusions to a solid support even under harshly denaturing conditions, such as high concentrations of guanidine hydrochloride and dithiothreitol. For such purposes, the preferred class of vector employs protein-RNA fusions. Such fusion proteins can be purified using oligonucleotide affinity columns with high specificity in the presence of chaotropic agents and strongly reducing conditions.

Under another preferred embodiment, other, non-bacterial, microbial systems, e.g., Pichia-based expression systems are employed. Kocken et al., Anal. Biochem. 239:111-112 (1996); Munshi et al., Protein Expr. Purif. 11: 104-110 (1997); Laroche et al., Bio/Technology 12:1119-1124 (1994) Cregg et al., Bio/Technology 11:905-910 (1993), all of which are herein incorporated by reference.

Once the protein domain of interest has been expressed at high levels, it is necessary to purify large quantities of the protein domain for subsequent characterization. Preferably, at least 5-10 mg of the protein domain of interests is purified. More preferably, at least 50 mg of the protein domain of interest is purified.

Methods for preparing large quantities of a given protein of sufficient purity for domain structure modeling are generally known to those of skill in the art. Although not all methods for protein purification are applicable to a given protein of interest, it is generally understood that the following methods represent preferred embodiments: affinity chromatography, ammonium sulfate precipitation, dialysis, FPLC chromatography, ion exchange chromatography, ultracentrifugation, etc. For a general review of protein purification methodologies, see Burgess, Protein Purification, In: Oxender et al. (Eds.), Protein Engineering, pp. 71-82, Liss (1987); Jakoby, (Ed.), Methods Enzymol. 104:Part C (1984); Scopes, Protein Purification: Principles and practice (2nd ed.), Springer-Verlag (1987), and related texts, all of which are herein incorporated by reference.

D. Rapid screening of NMR and crystallization properties

One common problem for both NMR analysis and crystallization is poor solubility and/or slow precipitation of the protein sample. These properties are highly dependent on the pH, ionic strength, reducing agent concentration, and other properties of the buffer solvent. Thus, it is preferable to optimize these conditions to maximize solubility for NMR analysis and to optimize the conditions for protein crystallization.

Under one of the preferred embodiments of the present invention, the optimization experiments are conducted with an array of microdialysis buttons to rapidly scan a plurality of standardized buffer conditions to identify those most suitable for NMR studies and/or crystallization of each domain construct (Bagby, J. Biomol. NMR 10:279-282 (1997), incorporated by reference in its entirety). Preferably, each microdialysis button contains at least 1 μL of a ˜1 mM protein solution. More preferably, each microdialysis button contains at least 5 μL of a ˜1 mM protein solution. The microdialysis buttons of the present invention are commercially available. Preferably, each microdialysis button is dialyzed against about 50 ml of dialysis buffer, such as in a 50 ml conical tube (Falcon). Preferably, the dialysis is performed at 4° C. However, the dialysis can be performed at temperatures ranging from 4°-40° C. Because NMR studies are routinely performed at room temperature for extended lengths of time, it is preferable that the protein remain in solution under these conditions.

Preferably, the protein samples are initially prepared in buffers containing 50% glycerol (which is not suitable for NMR studies but generally provides good solubility) and then dialyzed against different buffers containing little or no glycerol. With respect to NMR and X-ray crystallography studies, it is understood that a person of skill in the art would know what buffers could be used to prepare the protein for study. The skilled artisan typically has a set of 50-100 standard buffers which are used to prepare protein samples for subsequent studies. These buffers can then be modified if necessary to optimize the protein preparation. The ability of a given protein to remain soluble at high concentration or form suitable crystals is dependent on the pH of the solution, as well as the concentration of different salts, buffers, reagents, and temperature. Thus, the “button test” represents a preferred embodiment because it facilitates the rapid screening of a multitude of conditions.

This “button test” analysis typically requires 5-10 mg of protein sample and can be completed in a few days. Preferably, multiple samples are analyzed in parallel. Preferably, the protein samples are analyzed under a dissecting microscope to determine whether the protein has remained in solution or whether the protein has aggregated. Using the “button test” of the present invention, a single technician could score solubility properties in 100 different buffers for ˜20 domains per week. Under the another preferred embodiment, these screens can be carried out using state of the art laboratory automation technology.

Alternatively, the protein domain of interest is lyophilized and then resuspended in an appropriate buffer.

Having identified the conditions under which the protein domain of interest is soluble, dynamic light scattering can be used to examine its dispersive properties and aggregation tendency in different buffer conditions. Ferré-D'Amaré et al., Structure 15:357-359 (1994), herein incorporated by reference. Alternatively, Trp or Tyr fluorescence anisotropy can be used to measure rotational diffusion which is another measure of aggregation.

The “domain trapping” approach of the present invention includes an evaluation of NMR properties, and all of the protein samples which pass this stage of the process will already meet basic spectroscopic quality criteria. Standard criteria used to determine the basic spectroscopic quality of a given protein, which are known to those of skill in the art, include a good dispersion pattern and a narrow peak width, etc.

Preferably, gel filtration chromatography and dynamic light scattering data are collected during the course of domain purification. Such data provide information about the oligomerization state of the domain being studied.

For domains of the appropriate size (<˜30 kD), isotopically enriched samples are scored in terms of their suitability for structure determination by NMR using standard 2D HSQC, 2D NOESY, and/or 2D CBCANH triple-resonance spectra. The protein samples that provide good quality data for these NMR experiments are expected to provide good data in the full set of experiments required for automated structure determination. For each ¹⁵N, ¹³C enriched domain, this evaluation typically requires at least 5-10 mg of sample, and approximately 6 hours of NMR data collection. Preferably, the evaluation is performed on about 10 mg of sample. Thus, ˜20 domains can be evaluated per “spectrometer-week” using the methods of the present invention. A “spectrometer-week”, as used herein, means one skilled technician, working on one NMR machine would be able to evaluate approximately 20 domains in a given week.

Preferably, domains for structure determination by NMR are selected in an opportunistic manner, prioritizing those that provide high quality NMR data in the screens outlined above. Although some of the constructs that are generated may not be amenable to rapid structural analysis, it has been estimated that well over 50% of domains that are “trapped” by the process outlined above exhibit properties suitable for NMR or X-ray analysis. As these domains are derived from specific target genes associated with human diseases (discussed below) the chances of obtaining important new protein structures by this process are very high. Domains that provide diffraction quality crystals and which are not amenable to rapid analysis by NMR can be analyzed by X-ray crystallography.

E. Computer software and related NMR technologies for fully automated analysis of protein structures from NMR data

The present invention employs advanced NMR data collection and automated analysis technologies. These data collection and automated analysis technologies greatly accelerate the process of protein structure determination. Included within these technologies is a family of easy to use pulsed-field gradient triple resonance NMR experiments for rapid analysis of protein resonance assignments. See, for example, Montelione et al., Proc. Natl. Acad. Sci. (USA.) 86:1519-1523 (1989); Montelione et al., Biopolymers 32:327-334 (1992); Montelione et al., Biochemistry 31:236-249 (1992); Lyons et al., Biochemistry 32:7839-7845 (1993); Rios et al., J. Biomol. NMR 8:345-350 (1996); Tashiro et al., J. Mol. Biol. 272:573-590 (1997); Shimotakahara et al., Biochem. 36:6915-6929 (1997); Laity et al., Biochem. 36:12683-12699 (1997); Feng et al., Biochem. 37:10881-10896 (1998); and Swapana et al., J. Biomol. NMR 9:105-111 (1997), all of which are herein incorporated by reference. These data collection and automated analysis technologies further include a fully automated strategy for determining NMR resonance assignments in proteins. Zimmerman et al., Curr. Opin. Struct. Bio. 5:664-673 (1995); and Zimmerman et al., J. Mol. Biol. 269:592-610 (1997), both of which are herein incorporated by reference.

Preferably, the data collection and automated analysis technologies of the present invention employ multiple-quantum coherences in triple resonance for enhanced sensitivity. Swapna et al., J. Biomol. NMR 9:105-111 (1997); Shang et al., J. Amer. Chem. Soc. 119:9274-9278 (1997), both of which are herein incorporated by reference.

1. AUTOASSIGN: Artificial intelligence methods for automated analysis of protein resonance assignments

Resonance assignments form the basis for analysis of protein structure and dynamics by NMR (Wüthrich, K., NMR of Proteins and Nucleic Acids, John Wiley & Sons, New York, New York (1986), herein incorporated by reference) and their determination represents a primary bottleneck in protein solution structure analysis. However, the introduction of multi-dimensional triple-resonance NMR has dramatically improved the speed and reliability of the protein assignment process. Montelione et al., J. Magn. Res. 83:183-188 (1990); Ikura et al., Biochem. Pharmacol. 40:153-160 (1990); Ikura et al., FEBS Letters 266:155-158 (1990); Ikura et al., Biochem. 29:4659-4667 (1990), Tashiro et al., J. Mol. Biol. 272:573-590 (1997); Shimotakahara et al., Biochem. 36:6915-6929 (1997); Laity et al., Biochem. 36:12683-12699 (1997); Feng et al., Biochem. 37:10881-10896 (1998), all of which are herein incorporated by reference.

Preferably, the present invention employs AUTOASSIGN, an expert system that determines protein ¹⁵N, ¹³C, and ¹H resonance assignments from a set of three-dimensional NMR spectra. Zimmerman et al., Proceedings of the First International Conference of Intellegent Systems for Molcular Biology 1:447-455 (1993); Zimmerman et al., J. Biomol NMR 4:241-256 (1994); Zimmerman et al., Curr. Opin. Struct. Bio. 5:664-673 (1995); Zimmerman et al., J. Mol. Biol. 269:592-610 (1997), all of which are herein incorporated by reference. AUTOASSIGN has been copyrighted by Rutgers, the State University of New Jersey. Alternatively, the present invention can employ one of the following expert systems for the automated determination of protein ¹⁵N, ¹³C, and ¹H resonance assignments from a set of three-dimensional NMR spectra. These include a modified version of FELIX which is available from Molecular Simulation (San Diego, Calif.) (Friedrichs et al., J. Biomol. NMR 4:703-726 (1994), incorporated by reference in its entirety). CONTRAST which is available from the world wide web at <<www.bmrb.wisc.edu/macroo/soft_contrast.html>>(Olsen and Markley, J. Biomol. NMR 4:385-410 (1994), incorporated by reference in its entirety), and a series of small programs described by Meadows, J. Biomol. NWR 4:79-86 (1994), incorporated by reference in its entirety.

AUTOASSIGN is implemented in the Allegro Common Lisp Object System (CLOS) and requires a lisp compiler (available from Franz, Inc.) for execution. The software utilizes many of the analytical processes employed by NMR spectroscopists, including constraint-based reasoning and domain-specific knowledge-based methods. Fox et al., The Sixth Canadian Proceedings in Artificial Intelligence 1986); Nadel et al., Technical Report, DCS-TR-170, Computer Science Department, Rutgers Univ. (1986); Kumar et al., Artificial Intelligence Mag., Spring, 32-44 (1992), all of which are incorporated by reference in their entirety.

Input to AUTOASSIGN includes a peak-picked 2D (H-N)-HSQC spectrum and the following seven peak-picked 3D spectra: HNCO, CANH, CA(CO)NH, CBCANH, CBCA(CO)NH, H(CA)NH, and H(CA)(CO)NH. This family of triple-resonance experiments can be used together with AUTOASSIGN to automatically determine extensive sequence-specific ¹H, ¹⁵N, and ¹³C resonance assignments for several proteins ranging in size from 8 kD to 17 kD. Zimmerman et al., J. Mol. Biol. 269:592-610 (1997); Tashiro et al., J. Mol. Biol. 272:573-590 (1997); Shimotakahara et al., Biochem. 36:6915-6929 (1997); Laity et al., Biochem. 36:12683-12699 (1997); Feng et al., Biochem. 37:10881-10896 (1998). The program handles some of the very challenging problems encountered in automated analysis, including missing spin systems, spin systems that overlap even in the 3D spectra, and extra spin systems due to multiple conformations of the folded protein structure (e.g. X-Pro peptide bond cis/trans isomerization). Execution times on a Sun Sparc 10 workstation range from 16 to 360 sec, depending on the complexity of the problem analyzed by the program. Preferably, the NMR spectrometer of the present invention is equipped with three channels and a fourth frequency synthesizer for carbonyl decoupling. Under another preferred embodiment, the NMR spectrometer of the present invention is equipped with four channels.

In the present invention, the AUTOASSIGN program provides for automated analysis of resonance assignments for atoms of the polypeptide backbone. Preferably, the AUTOAS SIGN program of the present invention provides for fully automated analysis of resonance assignments. Having established assignments for the backbone atoms of each amino acid in the protein sequence, it is relatively straightforward to extend from these to sidechain ¹H and ¹³C resonance assignments using 3D HCCH COSY, HCCH-TOCSY, and HCC(CO)NH-TOCSY NMR experiments. Preferably, the AUTOASSIGN program of the present invention handles automated analysis of these sidechain resonance assignments. It is additionally preferred that 3D ¹⁵N-edited NOESY and 3D ¹³C-edited NOESY data are collected and automatically analyzed to confirm the resonance assignments.

Under one of the preferred embodiments of the present invention, AUTOAS SIGN is designed to implement strategies that allow complete resonance assignments to be obtained with fewer NMR spectra. For example, sensitivity enhanced versions of HCCNH-TOCSY and HCC(CO)NH-TOCSY experiments can provide the complete set of information required for the determination of resonance assignments. This reduces the total data collection time required for determining backbone resonance assignments from the current 7 - 10 days to about half of this time. Zimmerman et al., J. Biomol. NMR 4:241-256 (1994); Lyons et al., Biochemistry 32:7839-7845 (1993), both of which are herein incorporated by reference.

Perdeuteration greatly lengthens the ¹³C transverse relaxation rates, allowing for higher sensitivity in these triple-resonance experiments. Grzesiek et al., J. Biomol. NMR 3:487-493 (1993); Yamazaki et al., Eur. J. Biochem. 219:707-712 (1994), both of which are herein incorporated by reference. It has been demonstrated that significant sensitivity-enhancement (2 - 5 fold) can be obtained with triple-resonance experiments by perdeuteration of the protein samples. Preferably, the automated assignment strategy, described herein, will utilize ²H, ¹³C, ¹⁵N-enriched proteins prepared with protiated ¹⁵N-H amide groups, together with deuterium-decoupled triple resonance NMR experiments. Under one embodiment, the amide NH group in the perdeuterated protein exchanges rapidly with the solvent H₂O used in the course of the protein purification to yield the protiated ¹⁵N-H amide groups. This strategy can provide completely automated analysis of resonance assignments for the carbon and nitrogen skeleton of the protein. Having determined these assignments, analysis of resonance assignments for the attached hydrogen atoms can be completed using HCCH-COSY, HCCH-NOESY, and HCCH-TOCSY experiments. Correction factors for ²H-isotope shift effects for each carbon site of the 20 amino acids can be determined using data from model proteins. Preferably, the complete carbon resonance assignments in their protiated forms have already been determined for these model proteins.

Preferably, the present invention utilizes high temperature superconducting probes. First generation versions of these probes are currently being marketed by Varian NMR Inst. Inc. and Bruker Inst. Such probes in combination with the above-described technological advances reduce the time required for determining complete backbone and sidechain H, C, and N assignments to less than one week per domain.

2. Software for automated analysis of protein structures from NMR data

Having completed the resonance assignments for a particular protein, the next step of the structure determination process of the present invention involves analyzing secondary structure (i.e. α-helices, β-sheets, turns, etc.). The chemical shifts themselves are often sufficient to allow identification of these features of secondary structure in the protein. Spera, J. Amer. Chem. Soc. 113:5490-5492 (1991); Wishart et al., J. Biomol. NMR 6:135-140 (1995), both of which are herein incorporated by reference. This information can be combined with other bioinformatics data derived from the protein sequence to narrow the number of possible mappings of the protein to known chain folds, and possibly even to identify the protein's biochemical function.

The principal sources of information used for the structure determination of protein domains are nuclear Overhauser effect (NOE) data arising from magnetic dipole-dipole interactions between hydrogen atoms in the structure of the protein. Interpretation of these data from multidimensional NOE spectroscopy (NOESY) spectra requires the resonance assignments, which will be obtained (as described above) in an automated manner. Preferably, the present invention employs software for automated analysis of NOESY spectra and the generation of input files for rapid structure calculations using stimulated annealing of experimental constraint functions with molecular dynamics calculations.

The problems encountered in automatically analyzing NOESY spectra are due largely to spectral overlaps, i.e., it is often the case that several hydrogen atoms have very similar resonance frequencies. One of the preferred approaches to resolving this problem is to use 3D (or 4D) ¹⁵N- or ¹³C-resolved NOESY experiments (Clore et al., Ann. Rev. Biophys. Biophys. Chem. 20:29-63 (1991); Clore et al., Prog. Biophys. Mol. Bio. 62:153-184 (1994); Clore et al., Methods Enzymol. 239:349-363 (1994), all of which are herein incorporated by reference), in which one (or both) of the two protons involved in the NOE interaction is resolved in a third (or fourth) frequency dimension based on the frequency of the ¹⁵N or ¹³C nucleus to which it is covalently bound. Symmetry features of the 3D ¹³C-edited spectra can also be used to great advantage.

Another preferred approach to resolving ambiguities that arise in assigning NOESY cross peaks to specific pairs of interacting hydrogen atoms is to use the secondary structure (i.e. α helix, β strand, etc.) to predict NOEs that are expected and to use these structural predictions to guide the analysis of NOESY spectra. Meadows et al., J. Biomol. NMR 4:79-96 (1994), herein incorporated by reference.

A third preferred approach is to use a low-resolution structure of the protein obtained in a first pass analysis of the uniquely assigned NOESY cross peaks to identify candidate assignments of the remaining unassigned NOESY cross peaks which are inconsistent with the low-resolution structure.

The approaches outlined above are those that are routinely used by a human expert in the analysis of NOESY spectra. Under the preferred embodiment, the reasoning processes of those approaches are encoded into the software of the present invention. Preferably, the software program of the present invention is a C⁺⁺ program. AUTO_STRUCTURE is a C⁺⁺ program that analyzes 2D and 3D NOESY spectra to identify unique NOESY crosspeak assignments (Gaetano Montelione, Y. Huang and Robert Tejero (Rutgers, The State University of New Jersey)). The program then uses these crosspeak assignments to create distance-constraint input files for simulated annealing structure calculations. AUTO_STRUCTURE can also use a low-resolution (or homology-modeled) structure of the protein to filter the list of NOESY crosspeaks that are not uniquely assigned, removing potential NOE assignments that are severely inconsistent with the low-resolution structure. AUTO_STRUCTURE propagates the structural constraints imposed by the uniquely assigned NOEs to determine assignments of otherwise ambiguous NOEs. AUTO_STRUCTURE can successfully analyze NOESY spectra and, in an iterative fashion, automatically generate 3D structures of simple polypeptides. Other auto structure programs for NOESY analysis that can be used in the present invention include GARANT (Wüthrich (ETH, Zurcih, Germany), incorporated by reference in its entirety), ARIA (Michael Nilges, J. Mol. Biol. 245:645-660 (1995), incorporated by reference in its entirety) and NOAH (Mumenthaler and Braun, J. Mol. Bio. 254:465-420 (1995), incorporated by reference in its entirety).

Preferably, the auto structure program of the present invention provides for automated analysis of protein or protein domain structures. Under a more preferred embodiment, the auto structure program of the present invention further contains sophisticated reasoning processes which can assist in resolving ambiguous NOESY crosspeak assignments in the absence of even a low resolution 3D structure. Preferably, this includes (i) the propagation of structural constraint information inherent in the secondary structure analysis stemming from the resonance assignments and (ii) the application of pattern recognition algorithms.

F. Mapping new domain structures to proteins in the Protein Data Base (PDB) with similar structures and biochemical functions

Preferably, the resulting domain structures derived from NMR or X-ray crystallographic analyses are compared with the PDB or other suitable databases of known protein structures using an algorithm for 3D-structure homology matching. Examples of publicly available PDBs suitable for use in the present invention include the Protein Data Base (PDB), which can be found at http://www.pdb.bnl.gov/. Algorithms for 3D-structure homology matching suitable for use in the present invention include the DALI analysis program (Holm et al., J. Mol. Biol. 233:123-138 (1993), herein incorporated by reference), the CATH analysis program (Orengo, C. A., Structure 5:1093-1108 (1997), herein incorporated by reference), VAST (http://www.ncbi.nlm.nih.gov/Structure/vast.html; Gibrat et al., Current Opinion in Structural Biology 6: 377-385 (1996); and Madej et al., Proteins 23: 356-369 (1995), all of which are incorporated by reference in their entirety) or similar algorithms for 3D-structure homology matching.

DALI compares “contact maps” of protein structures to identify homologies in 3D structure and provides a list of PDB entries with high match scores. Based on current “hit” rates by newly-determined structures against already known folds (Holm et al., Methods Enzymol. 266:653-662 (1996); Holm et al., Science 273:595-603 (1996), both of which are herein incorporated by reference), it is expect that greater than 50% of the structures will show significant structural and functional homology to proteins of known structure and function.

In order to facilitate and enhance the ability to identify common biochemical functions for these DALI hits, it is preferable to develop a structure-function knowledge base (FIG. 1), correlating each protein structure in the PDB with the set of biochemical functions that have been associated with that protein in the published scientific literature. Where information is available, this knowledge base will also correlate the portions of these known protein structures with corresponding specific biochemical functions (e.g., enzymatic active sites or nucleic-acid binding loops). This fold-function knowledge base is applicable to a wide range of structural bioinformatics applications, and of significant utility to the nascent industry of structural bioinformatics.

Once novel protein domains with clear homologies to better-characterized counterparts have been identified, the proposed functions can be validated using biochemical assays. For example, if a protein looks like a member of the galactosyl transferase family, the protein will be tested for radioactive UDP-galactose (or other carbohydrate) binding, if it looks like a lipase, the protein will be tested for lipid binding and/or hydrolysis activity, and so on.

G. Integration into a large-scale, high-throughput “engine” for structural and functional analysis of hundreds of human genes

Under one preferred embodiment, the present invention provides for a “structure function analysis engine” capable of high-throughput discovery of biochemical functions of new human disease genes and genes of unknown function.

Using conventional methodology, the skilled artisan may be able to determine the 3D structure of one protein per year. However, using the methodology of the present invention, it is possible to determine the 3D structure of far greater than one protein per year. Under optimal conditions, the present invention will enable a properly equipped laboratory to generate the 3D structure of one protein per month per NMR machine. As used herein, “high-throughput” refers to the ability to determine the 3D structures of protein domains of unknown function at a rate which is faster than the rate at which a skilled artisan could determine a protein structure using traditional methodologies.

One of the central features of the present invention is that it is highly scaleable. Under one of the preferred embodiments, the high-throughput “engine” consists of a dedicated laboratory staffed with artisans skilled in relevant arts (e.g., NMR and X-Ray crystallography, molecular biology, biochemistry, etc.). Preferably, such a laboratory is further equipped with state of the art equipment for the sequencing, sub-cloning, expression, purification, screening and analysis of the protein domains of interest. The rate limiting component of this high-throughput “engine” is the number of NMR machines within the laboratory. Thus, the rate at which protein domains can be characterized will increase with the addition of additional NMR machines. Unlike conventional methodology, the present invention provides a method for determining the 3D structure of unknown protein domains whose rate is not solely dependent on the number of artisans skilled in 3D protein structure determination.

The rate of domain characterization increases as each of the tasks which are presently conducted by hand are automated. For example, under one of the preferred embodiments, the parsing of the unknown gene into its component domains is facilitated through the use of advanced sequence analysis algorithms. Under another of the preferred embodiments, the rate of domain characterization is increased through the use of improved computer software for the automated analysis of NMR datapoints.

Although the present invention is drawn to using NMR to determine protein structure and function, it is to be understood that a person of skill in the art could perform similar analysis using X-ray crystallography to practice the present invention. Shapiro and Lima, J. Structure 6:265-267 (1998); Gaasterland, Nature Biotech. 16:625-627 (1998); Terwilliger et al. Prot. Sci. 7:1851-1856 (1998); Kim, Nature Structure Biology (Synchrotron Supp.): 643-645 (1998), all of which are incorporated by reference in their entirety.

V. SPECIFIC GENE TARGETS

Preferably, the specific gene targets that will be analyzed using the present invention will be genes that are known to be involved in human diseases but for which the biochemical function and three-dimensional structures of the proteins encoded by the genes are not available. These protein domains will be analyzed using the high-throughput “structure—function analysis engine” of the present invention. The resulting structural and functional information will be critical in developing pharmaceuticals targeted to these human gene products.

Although the present invention is principally drawn to human genomic, cDNA and mRNA sequences, it is to be understood that the present invention is generically applicable to genomic, cDNA and mRNA sequences of any living organism or virus.

Although the present invention is capable of determining the function of any given protein or protein domain, the preferred biomedical gene targets of the present invention include Alzheimer's β peptide precursor protein (APP). Additional preferred biomedical gene targets include but are not limited to those genes implicated in neoplastic, neurodegenerative, metabolic, cardiovascular, psychiatric and inflammatory disorders. The genomes/genes of infectious agents, such as pathogenic microbes, pathogenic fungi and pathogenic viruses, are also preferred targets for study.

By focusing on medically important diseases, it is anticipated that the present invention will greatly facilitate the identification of protein targets for subsequent drug discovery efforts.

Having now generally described the invention, the same will be more readily understood through reference to the following examples which are provided by way of illustration and are not intended to be limiting on the present invention.

### EXAMPLE 1 PARSING OF THE APP GENE INTO DOMAIN-ENCODING REGIONS

A. Parsing by the exon phase rule The human amyloid beta peptide precursor (APP) protein gene (Yoshikai et al., Gene 87:257-263(1990)) was subjected to a parsing analysis with respect to the phases of its exon-exon boundaries:

| Exon-exon boundary | Phase |
|---|---|
| 1-2 | 0 |
| 2-3 | 0 |
| 3-4 | 1 |
| 4-5 | 0 |
| 5-6 | 2 |
| 6-7 | 1 |
| 7-8 | 1 |
| 8-9 | 1 |
| 9-10 | 0 |
| 10-11 | 0 |
| 11-12 | 0 |
| 12-13 | 0 |
| 13-14 | 1 |
| 14-15 | 1 |
| 15-16 | 1 |
| 16-17 | 0 |
| 17-18 | 0 |

Using the exon phase rule, only exons or exon combinations that start or stop in the same phase are allowed. For example, exon 7 or exons 7+8 are potential domain encoding regions with phase 1 boundaries. Likewise, exon 10, exons 10+11, and exons 10+11+12 would be potential domain encoding regions with phase 0 boundaries.

B. Exon phase and the alternative splicing rule

The APP gene is reported to be alternatively spliced. The longest polypeptide encoded by the APP gene is 770 amino acids long, and shorter isoforms exist that are missing the amino acids encoded by exons 7, 8, and/or 15 (Sandbrink et al., Ann. NY Acad. Sci. 777:281-287 (1996), herein incorporated by reference). All of these exons which are alternatively spliced are bounded by phase 1 termini. Alternative splicing must be done in such a way as to not disrupt the integrity of the holoprotein (i.e., without destroying essential folding information). The fact that all alternatively spliced exons have phase 1 termini implies that domain boundaries may be congruent with phase 1 exon boundaries, that is, phase 1 exon boundaries in this particular gene are candidate boundaries of domain encoding regions.

C. Setting the phase with known internal domain structures

Exon 7 of APP is known to encode a complete domain for a Kunitz-type serine protease inhibitor (Hynes et al., Biochemistry 29:10018-10022 (1990)). The Kunitz inhibitor is a domain that has been combinatorially shuffled around in various genes during evolution (Patty, L. Curr. Opin. Struct. Biol. 1:351-361 (1991)), and for the reasons given above it would have to be inserted only into proteins with other domains of the same phase in order to not disrupt gene expression. Therefore, this analysis is also consistent with APP being composed of domains which are bounded by phase 1 exon termini.

D. The “N-terminus first” strategy of parsing

In order to reduce the combinatorial complexity of the parsing problems, an “N-terminus first” strategy is preferred. In this parsing strategy, expression constructs of putative domains are made starting from the N-terminus of the protein and extending to the likely C-termini as predicted by the above rules. These constructs are put through the “domain trapping” test of the present invention in order to identify the first N-terminal domain. Then, once the first N-terminal domain is identified, a second set of constructs commencing from the C-terminus of the first N-terminal domain is made, and so on.

In the case of APP, the N-terminus of the protein starts with exon 2 because exon 1 encodes a signal peptide. Therefore, the possible domain constructs that ended in phase 1 boundaries were exons 2-3 and exons 2-6 (exon 7 was known to encode the Kunitz inhibitor domain). By the domain trapping criteria exons 2-3 were found to encode the first N-terminal domain, so a second construct composed of exons 4-6 was made and found to contain the second domain of APP, and so on. A summary of the APP domains identified by this combination of parsing and domain trapping is given below:

| Domain | Encoding Exons |
|---|---|
| 1 (N-terminal domain) | 2-3 |
| 2 | 4-6 |
| 3 (Kunitiz inhibitor) | 7 |
| 4 | 8 |
| etc. | |

### EXAMPLE 2 EXPRESSION AND PURIFICATION OF AN ISOLATED DOMAIN

The putative domain regions identified in Example 1 are sub-cloned into the secretion-based protein A fusion expression system and purified. Nilsson et al., Methods Enzymol. 185:144-161 (1990), herein incorporated by reference.

### EXAMPLE 3 EXPRESSION AND PURIFICATION OF AN ISOLATED DOMAIN FOR NMR ANALYSIS

Protein Expression

E. coli strain RV308 is used as the bacterial expression host. Competent RV308 cells are transformed with pHAZY plasmid containing the NTD 2-3, Z domain insert. Cells are grown overnight at 37° C. on LB agar plates supplemented with 100 g/ml ampicillin (Sigma). Fresh transformants are used to inoculate seed cultures in 2×TY media (16 g/l typtone, 10 g/l yeast extract, and 5/g NaCl) supplemented with 100 μg/ml ampicillin. Cultures are grown overnight at 30° C. in 250 ml baffled flasks. A ratio of 1 to 25 is used to inoculate expression cultures. For 1 liter of MJ media expression culture (2.5 g/l ¹⁵NH₄ sulfate (>98% purity), 0.5 g/l sodium citrate, 100 mM potassium phosphate buffer, pH 6.6, supplemented with 5 g/l ¹³C-glucose (>98% purity), 1 g/l magnesium sulfate, 70 mg/1 thiamine, 1 ml of 1000×trace elements solution, 1 ml of 1000×vitamin solution, and 100 mg/1 ampicillin), 40 ml of seed culture is spun down by centrifugation. Bacterial pellets are washed, resuspended in fresh MJ media, and used to inoculate expression cultures. Cultures are grown at 30° in 2 1 baffled flasks and induced at OD⁵⁵ 0.9-1.0 with indole acrylic acid to a final concentration of 20 mg/l. Cultures are harvested 15 hours after induction by centrifugation. Bacterial pellets are stored at 20° C. until purification.

Protein Purification

Bacterial cells are resuspended in 100 ml of 25 mM Tris, pH 8.0, 5 mM EDTA, 0.5% Triton X-100 and sonicated continuously for 9 minutes. Released inclusion bodies are pelleted by centrifugation and washed with fresh sonication buffer. Inclusion bodies were then solubilized with 7 M guanidine HCl and 10 mM DTT. Centrifugation is used to pellet any undissolved material. Guanidine and DTT are then diluted twenty fold by dialysis against twenty volumes of 10 mM HCl.

IgG affinity purification is used to purify the NTD 2-3, Z domain fusion from any contaminating proteins. The 10 mM HCl protein solution is neutralized to >pH 7 with 1 M Tris, pH 8.0. The sample is then applied to an IgG sepharose column (Pharmacia) pre-equilibrated with TST buffer. The column is washed with 10 bed volumes of TST (50 mM Tris, 150 mM NaCl, and 0.05% TWEEN™20) followed by 2 bed volumes of 5 mM ammonium acetate, pH 5.0. Finally, the protein is eluted with 0.5 M acetic acid, pH 3.4. In preparation for refolding, the protein eluate is neutralized to pH 8.0 with solid Tris, and an equal volume of 7 M guanidine is added to bring the final guanidine concentration to 3.5 M.

Refolding of the protein is carried out by using dialysis to slowly dilute out the guanidine HCl while slowly introducing the refolding buffer. Firstly, Spectra/POR dialysis tubing with a MWCO of 6000-8000 is soaked overnight in water in order to remove glycerol. Next, the protein solution is loaded into the primed tubing and dialyzed against fresh refolding buffer. The dialysis reaction is incubated for two days at 4° C. with magnetic stirring. Refolded protein is then concentrated using an IgG sepharose column pre-equilibrated with TST buffer. Bound protein is eluted with 0.5 M acetic acid and collected in fractions in order to keep the volume as low as possible. Refolded fusion protein is then further purified by gel filtration on a Pharmacia Superdex 75 FPLC column using 300 mM ammonium bicarbonate, 0.1 mM copper sulfate as the buffer. Fractions corresponding to the fusion protein are pooled, and the protein is quantitated using the optical density at 280 nm.

Cleavage of the fusion protein is carried out using Genenase I (NEB), a variant of subtilisin BPN′. Fusion protein is buffer exchanged into Genenase buffer, 20 mM Tris, pH 8.0, 200 mM NaCl, 0.02% NaN₃, using an Amicon stir cell. The protein concentration is adjusted to 2 mg/ml and Genenase is added to a concentration of 0.2 mg/ml. The reaction is incubated at room temperature for 4 days and the extent of cleavage was followed using SDS-PAGE. Cleaved NTD 2-3 is separated from uncleaved fusion and Z domain by passing the solution over an IgG column and collecting the unbound NTD 2-3 in the flow through. The NTD is then purified from Genenase by gel filtration on a Pharmacia Superdex 75 FPLC column using 300 mM ammonium bicarbonate, 0.1 mM copper sulfate as the buffer.

### EXAMPLE 4 DOMAIN TRAPPING:CHARACTERIZATION OF AN ISOLATED DOMAIN

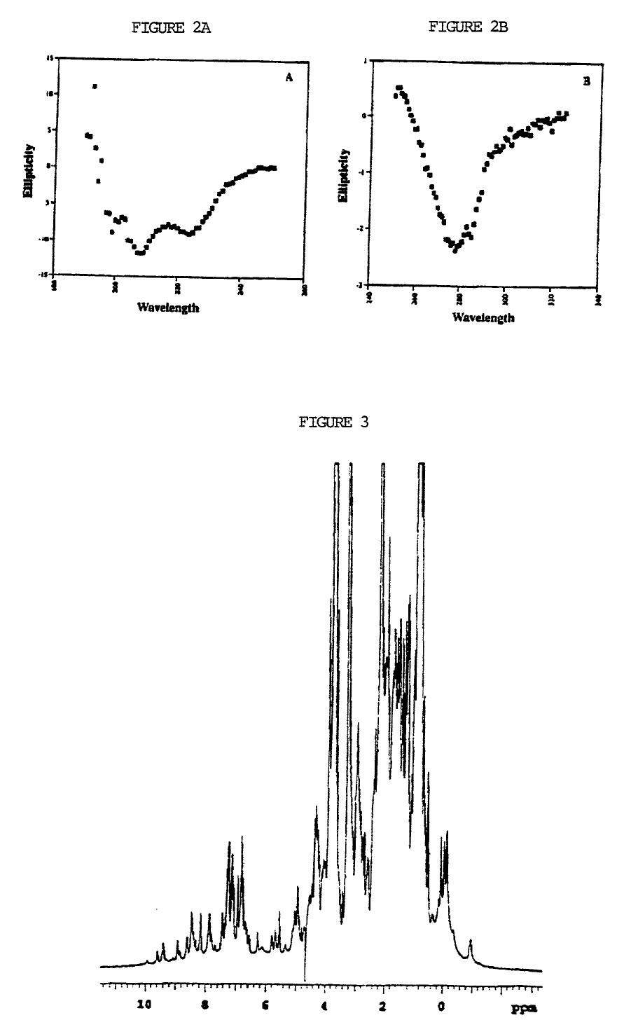

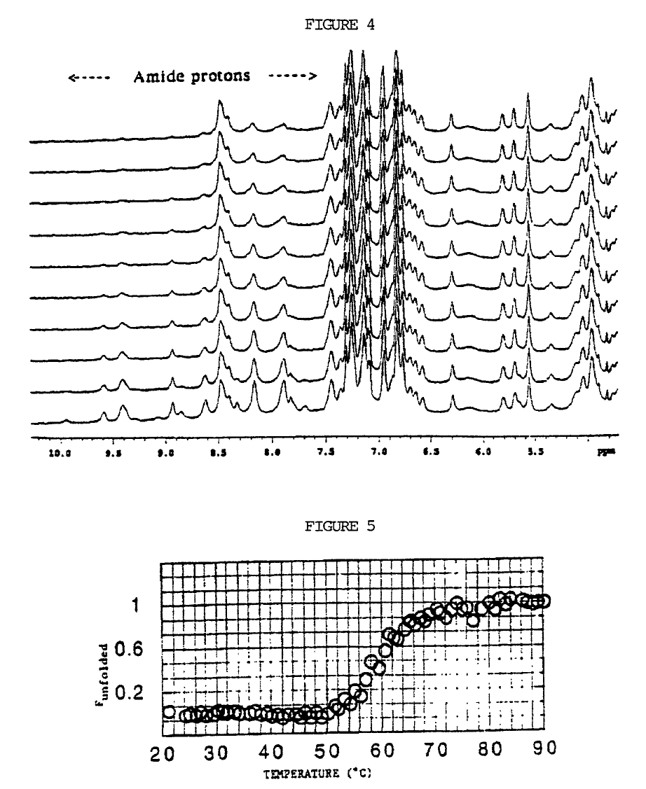

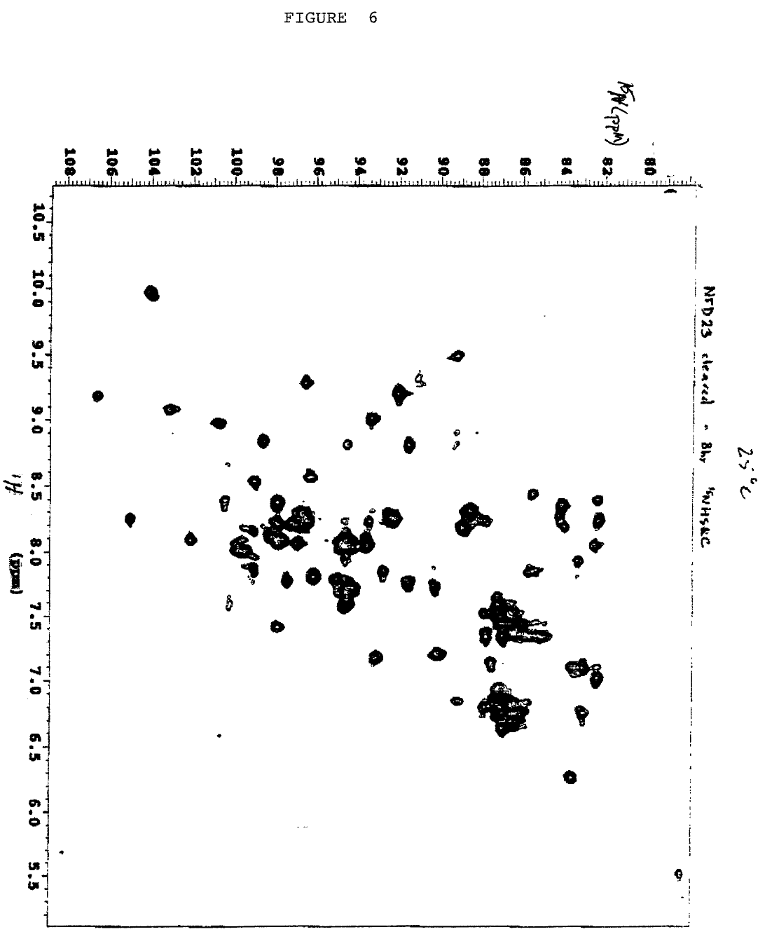

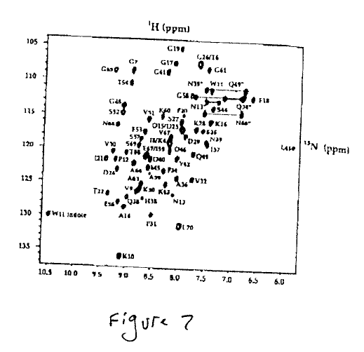

Characterization of an isolated domain (NTD2-3) from the Alzheimer's amyloid precursor protein (APP) by circular dichroism measurements in the far UV shows an ellipticity minimum at 222 nm, indicative of ax-helical secondary structure (FIG. 2A). Of even greater significance, CD measurements at longer wavelengths reveal a clear signal in the aromatic region around 280 nm, consistent with the presence of Trp, Tyr, and Phe chromophores in an ordered environment such as would be expected in the hydrophobic core of a folded protein (FIG. 2B). A moderately concentrated solution (˜100 μM) of the isolated N-terminal domain is further characterized by one-dimensional ¹H-NMR. The isolated recombinant APP N-terminal domain exhibits high dispersion of the proton resonances, which is a signature of well-folded polypeptides (FIG. 3).

A time-course of amide hydrogen-deuterium exchange measurements is performed. From this, it is observed that many backbone NH groups exhibit significant protection, indicating hydrogen-bonded secondary structure stabilized by tertiary interactions consistent with a well-folded domain structure (FIG. 4). Finally, thermal denaturation experiments, monitored by intrinsic tryptophan fluorescence, are performed. These experiments show that the recombinant APP NTD2-3 domain undergoes a cooperative thermal unfolding transition, with a Tₘ of approximately 60° C., indicative of a compact domain structure (FIG. 5).

Thus, using biophysical data alone, it is demonstrated that the NTD2-3 domain of APP, encoded by exons 2 and 3, is expressed as a well ordered tertiary structure. Chiang et al., Neurobiol. Aging, Supplement Vol. 17, No. 4S, abstract 393 (1996). Similar studies indicate that the next APP N-terminal domain is encoded by exons 4-6, the third (Kunitz) domain by exon 7, and so on.

### EXAMPLE 5 NMR CHARACTERIZATION OF THE NTD 2-3 DOMAIN

For NMR studies NTD 2-3 is concentrated to concentrations greater than 10 mg/ml. Gel filtration pure NTD 2-3 is first buffer exchanged into a NMR compatible buffer, 20 mM potassium phosphate, pH 6.5 using an Amicon stir cell. The protein solution is then concentrated to an appropriate volume based on the amount of protein present using the Amicon 50 and Amicon 3 stir cells. The final protein concentration is confirmed by optical density at 280 nm.

NMR ¹⁵N-HSQC spectra is collected on a Varian Unity 500 spectrometer. The ¹⁵N-HSQC spectral analysis is shown in FIG. 6. The good dispersion in both the ¹⁵N and ¹H dimensions demonstrate that this is a folded domain that has been “trapped” by the presently described methods.

### EXAMPLE 5 COMPARISON OF THE NMR STRUCTURE OF CSPA WITH OTHER PROTEINS

Recombinant CspA is expressed and purified using the protocol essentially as described by Chatterjee et al., J. Biochem. 114:663-669 (1993), and Feng et al., Biochemistry 37:10881-10896 (1998), both of which are incorporated by reference in their entirety. The purified CspA protein is prepared for NMR analysis by dialysis against a buffer containing 50 mM potassium phosphate and 1 mM NaN₃, pH 6.0 and the sample is analyzed using a Varian Unity 500 spectrometer equipped with three channels and a fourth frequency synthesizer for carbonyl decoupling as described by Feng et al., Biochemistry 37:10881-10896 (1998). FIG. 7 provides the 2D ¹⁵N-¹H^{N} HSQC spectrum of CSPA at pH 6.0 and 30° C.

The collected spin resonances are analyzed using AUTOASSIGN. The input for AUTOASSIGN includes peaks from 2D ¹⁵N-¹H^{N} HSQC and 3D HNCO spectra along with peak lists from three intraresidue (CANH, CBCANH and HCANH) and three interresidue (CA(CO)NH, CBCA(CO)NH and HCA(CO)NH) experiments, which correlate with the C^{α}, C^{δ} and H^{α} resonances of residues i and i-1 respectively. The results of the AUTOAS SIGN analysis of the peak picked 2D and 3D NMR spectra are summarized in Table 1.

Side chain resonance assignments are obtained using PFG HCCNH-TOCSY and PFG HCC(CO)NH-TOCSY and homonuclear TOCSY experiments recorded with multiple mixing times of 22, 36, 45, 54, 71 and 90 ms according to the method of Celda and Montelione, J. Magn. Reson. B]01:189-193 (1993), incorporated by reference in its entirety. Interatomic distance constraints are derived from three NOESY data sets 2D NOESY and 3D ¹⁵N-edited NOESY-HSQC spectra recorded with a mixing time of tₘ of 60 ms of a CspA sample dissolved in 90% H₂O/10% ²H₂O and a 2D NOESY spectrum is recorded with a mixing time tₘ of 50 ms of a sample dissolved in 100% ²H₂O. The intensity of the NOESY-HSQC spectrum is corrected for ¹⁵N relaxation effects, and the cross-peak intensities are converted into interproton distance constraints.

TABLE 1
Summary of AUTOSSIGN Analysis for
CspA Triple-Resonance NMR Data

| | | Number of | | |
|---|---|---|---|---|
| | | assignments | AUTOSSIGN | Manual |
| Residues | 69 | (expected) | analysis | analysis |
| | | Backbone | | |
| GSs expected | 66 | H^{N} | 65 | 66 |
| GSs observed | 67 | H^{α} | 77 | 79 |
| Degenerate | 8 | ¹⁵N | 65 | 66 |
| GS roots | | | | |
| Assigned GSs | 65 | ¹³C^{α} | 67 | 69 |
| Extra GSs | 2 | ¹³C^{δ} | 64 | 66 |
| Assigned | 68 | ¹³C^{β} | 49 | 59 |
| residues | | | | |
| Percent | 99% | Side chain | | |
| assigned | | | | |
| residues | | | | |
| Execution | 10 | ¹⁵N | 6 | 6 |
| times | | | | |
| (sec.) | | | | |
| | | H^{N} | 11 | 11 |

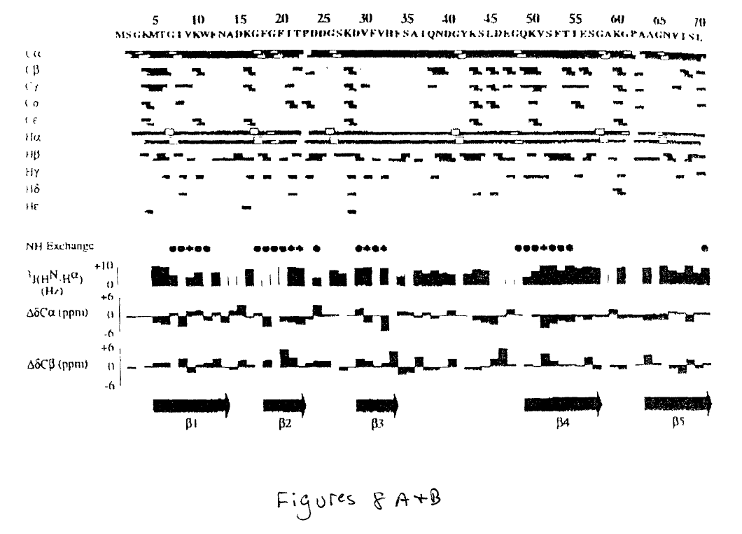

Stereospecific assignments of methylene H^{β}s are made by analysis of local NOE and vicinal coupling constant data using the HYPER program. HYPER is a conformational grid search program used for determining stereospecific C^{β}H₂ methylene proton assignments and for defining the ranges of dihedral angles φ, ψ, χ¹ that are consistent with the local experimental NMR data for each amino acid in a polypeptide (Tejero et al., J. Biomol. NMR (in press), incorporated by reference in its entirety). The secondary structural elements of CspA are summarized in FIG. 8. From this information, five β-strands corresponding to polypeptide segments of residue 5-13, 18-22, 30-33, 50-56 and 63-70 are identified.

The average number of distance contraints per residue is 10.4. Dihedral angel constraints are obtained from the HYPER program. Structure generation calculations are carried out with DIANA, version 2.8 TRIPOS, Inc.) using R8000 processor in a Silicon Graphics Onyx workstation (Braun and Go, J. Mol. Biol. 186:611-626 (1985), and Guntert et al., J. Mol. Biol. 169:949-961 (1983), both of which are incorporated by reference in their entirety).

From this NMR data set, the solution structure of CspA is reasonably well defined. Using the refined CspA coordinates defined by the present invention, structural database searches of the Protein Data Base (PDB) are performed with the DALI program. This search is able to identify a list of proteins or domains of structural homologues. Identified structural homologues of CspA exhibiting similar biochemical function include the RNA binding domain of E. coli polyribonucleotide nucleotidyltransferase, the human mitochondrial ssDNA-binding protein, E. coli translation initiation factor 1, the ssDNA-binding protein from gene V of filamentous bacteriophages M13 and f1, the ssDNA-binding protein from Pseudomonas phage Pf3, elongation factor G from Thermus thermophilus, a domain of E. coli lysyl tRNA synthetase, a domain of yeast tRNA synthetase, human replication protein A, staphylococcus nuclease, and a domain of E. coli topoisomerase I. Although the function of CspA was already know, the present Example has illustrated the use of the present invention.

As the present invention describes, a person of skill in the art is able to take a polypeptide of unknown function, express and purify a stable peptide domain encoded by the polypeptide, determine the NMR 3D structure of that expressed domain and predict the function of that domain by comparing the structure of that domain against known structures having known functions. This represents a fundamental paradigm shift in the study of proteins.

It will be apparent to those skilled in the art that various modifications may be made in the present invention without departing from the spirit and scope of the present invention. It will be additionally apparent to those skilled in the art that the basic construction of the present invention is intended to cover any variations, uses or adaptations of the invention following, in general, the principle of the invention and including such departures from the present disclosure as come within known or customary practice within the art to which the invention pertains. Therefore, it will be appreciated that the scope of this invention is to be defined by the claims appended hereto, rather than the specific embodiments which have been presented as examples.

We claim:

1. A high-throughput method for determining a biochemical function of a protein or polypeptide domain of unknown function comprising:
(A) identifying a putative polypeptide domain that properly folds into a stable polypeptide domain, said stable polypeptide having a defined three dimensional structure;
(B) determining three dimensional structure of the stable polypeptide domain;
(C) comparing the determined three dimensional structure of the stable polypeptide domain to known three-dimensional structures in a protein data bank, wherein said comparison identifies known structures within said protein data bank that are homologous to the determined three dimensional structure; and
(D) correlating a biochemical function corresponding to the identified homologous structure to a biochemical function for the stable polypeptide domain.

2. The method according to claim 1, further comprising the prestep of parsing a target polynucleotide into at least one putative polypeptide domain.

3. The method according to claim 2, wherein said parsing is performed by a first computer algorithm, wherein said first computer algorithm is selected from the group consisting of a computer algorithm capable of determining exon phase boundaries of a polynucleotide, and a computer algorithm capable of determining interdomain boundaries encoded in a polynucleotide.

4. The method of claim 3, further comprising a computer algorithm that compares the putative polypeptide domain sequence with known domain sequences stored within a database.

5. The method of claim 1, wherein said identification of the stable polypeptide domain having a defined three dimensional structure is performed by a set of activity-independent biophysical criteria that assesses the correctness of folding of the polypeptide domain, said set of activity-independent biophysical criteria including at least one of the criteria selected from the group consisting of circular dichroism measurements, ¹H-NMR spectroscopy, amide hydrogen-deuterium time course exchange, and thermal denaturation.

6. The method of claim 1, wherein said determination of the three dimensional structure of the stable polypeptide domain is obtained from an NMR spectrometer spectra of said polypeptide domain.

7. The method of claim 6, wherein said NMR spectrometer spectra include one or more spectra selected from the group consisting of nuclear Overhauser effect spectroscopy (NOESY), pulsed-field gradient ¹⁵N-heteronuclear single-quantum coherence spectroscopy (PFG-HSQC), pulsed-field gradient triple-resonance HCCNH ¹³C-¹³C total correlation spectroscopy (PFG-HCCNH-TOCSY), pulsed-field gradient HCC(CO)NH ¹³C-¹³C TOCSY (PFG-HCC(CO)NH-TOCSY), HCCH COSY, HCCNH-TOCSY, HNCO, CANH, CA(CO)NH, CBCNH, CBCA(CO)NH, H(CA)NH, and H(CA)(CO)NH.

8. The method of claim 6, wherein said NMR spectra is analyzed by a second computer algorithm that automatically assigns resonance assignments to the polypeptide sequence.

9. The method of claim 1, wherein said identification of said stable polypeptide domain comprises measuring a time course of amide hydrogen-deuterium exchange.

10. The method of claim 1, wherein prior to step (B), said stable polypeptide domain is optimally solubilized, said optimum solubilization comprising:
i) preparing an array of microdialysis buttons, wherein each of said microdialysis buttons contains at least 1 μl of an approximately 1 M solution of said stable polypeptide domain;
ii) dialyzing each member of said array of microdialysis buttons against a different dialysis buffer;
iii) analyzing each of said dialyzed microdialysis buttons to determine whether said stable polypeptide domain has remained soluble; and
iv) selecting the polypeptide domain having optimum solubility characteristics for NMR spectroscopy.

11. The method of claim 1, wherein said comparison of said determined three dimensional structure to said known three-dimensional structures in the protein data bank is performed by a third computer algorithm that is capable of determining 3D structure homology between said determined three dimensional structure and a member of said PDB.

12. The method according to claim 11, wherein said third computer algorithm is selected from the group consisting of DALI, CATH and VAST.

13. The method of claim 1, wherein said protein data bank is Protein Data Base (“PDB”).

14. The method of claim 4, wherein said database contains domain sequence information of known and determined domain sequences.

15. An integrated system for rapid determination of a biochemical function of a protein or protein domain of unknown function:
(A) a first computer algorithm capable of parsing said target polynucleotide into at least one putative domain encoding region;
(B) a designated lab for expressing said putative domain;
(C) an NMR spectrometer for determining individual spin resonances of amino acids of said putative domain;
(D) a data collection device capable of collecting NMR spectral date, wherein said data collection device is operatively coupled to said NMR spectrometer;
(E) at least one computer;
(F) a second computer algorithm capable of assigning individual spin resonances to individual amino acids of a polypeptide;
(G) a third computer algorithm capable of determining tertiary structure of a polypeptide, wherein said polypeptide has had resonances assigned to individual amino acids of said polypeptide;
(H) a database, wherein stored within said database is information about the structure and function of known proteins and determined proteins; and
(I) a fourth computer algorithm capable of determining 3D structure homology between the determined three-dimensional structure of a polypeptide of unknown function to three-dimensional structure of a protein of known function, wherein said protein of known structure is stored within said protein database.

16. The integrated system of claim 15, wherein said fourth computer algorithm is selected from the group consisting of DALI, CATH and VAST.

17. A high-throughput method for determining a biochemical function of a polypeptide of unknown function encoded by a target polynucleotide comprising the steps:
(A) identifying at least one putative polypeptide domain encoding region of the target polynucleotide (“parsing”);
(B) expressing said putative polypeptide domain;
(C) determining whether said expressed putative polypeptide domain forms a stable polypeptide domain having a defined three dimensional structure (“trapping”);
(D) determining the three dimensional structure of the stable polypeptide domain;
(E) comparing the determined three dimensional structure of the stable polypeptide domain to known three dimensional structures in a Protein Data Bank to determine whether any such known structures are homologous to the determined structure; and
(F) correlating a biochemical function corresponding to the homologous structure to a biochemical function for the stable polypeptide domain.
